(19) United States
(12) Plant Patent Application Publication
(10) Pub. No.: US 2003/0217399 P1
(43) Pub. Date: Nov. 20, 2003

(54) Prunus plant named 'VSV-1'

(76) Inventor: Guennadi Eremin, Krymsk (RU)

Correspondence Address: Ms. Shannon K. Reeser, Varieties International, P.O. Box 753, Lafayette, OR, 97127, US

(21) Appl. No.: 09/880,951

(22) Filed: Jan. 11, 2002

(51) Int. Cl. A01H 5/00

(52) U.S. Cl. PLT/180

(57) ABSTRACT

A new and distinct rootstock variety of Prunus named ‘VSV-1’ that is characterized by, vigor and strength, the ability to serve well as rootstock that is compatible with peach and plum, resistance to drought, cold and heat and the ability to propagate well by stoolbeds in addition to other methods. In combination these traits set the new cultivar apart from all other existing varieties of Prunus rootstock.

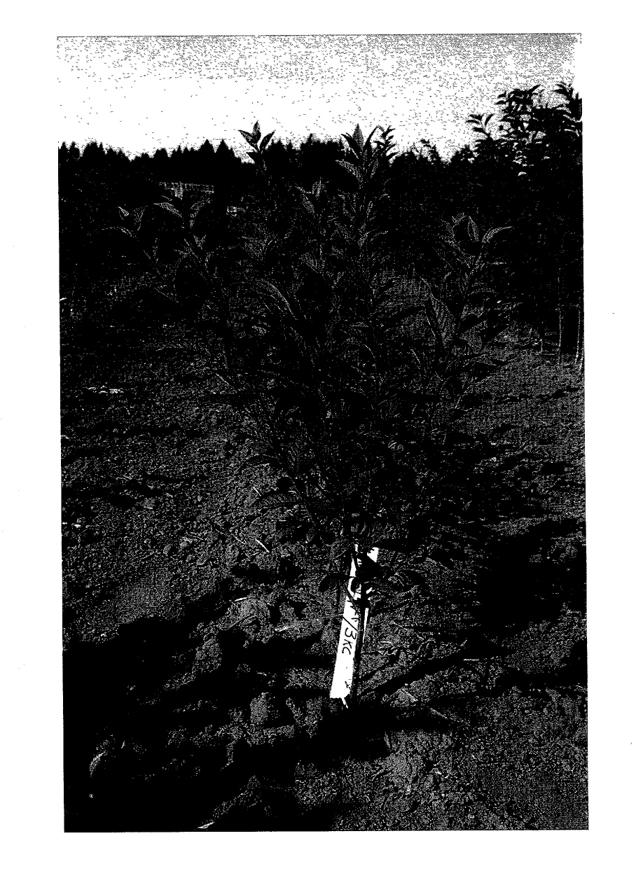

### CROSS-REFERENCES TO RELATED APPLICATIONS

The application for the new invention Prunus Plant Named ‘VSV-1’ will be co-pending with three other applications entitled Prunus Plant Named ‘VVA-1’, Prunus Plant Named ‘VSL-2’, and Prunus Plant Named ‘LC-52’ having the same filing date and inventor.

### BACKGROUND OF THE INVENTION

The present invention relates to the new and distinct cultivar known botanically as a hybrid of Prunus and referred to hereinafter as ‘VSV-1’. The new invention was bred by the inventor at the Breeding Station in Krymsk, Russia.

The breeding program at the Breeding Station was established in 1966 and funded by the government of the former Soviet Union for the purpose of producing new and improved Prunus cultivars that propagate well, have one central stem, few branches, and serve well as rootstock that is compatible with peach and plum.

In 1966 the inventor crossed the female parent Prunus incanus (not patented) with the male parent Prunus tomentosa (not patented), producing an induced hybridization in a cultivated area of Krymsk, Russia. The resulting seedlings were observed and evaluated for ten years. In 1977 the inventor selected ‘VSV-1’ from these seedlings. The new cultivar is the result of a hybrid cross between the Prunus incana (not patented) and Prunus tomentosa (not patented).

The closest comparison plants are the parent plants. The characteristics that distinguish the new cultivar from Prunus incana are, increased vigor, larger fruit, larger leaves and ease of propagation. The characteristics that distinguish the new cultivar from Prunus tomentosa are, narrow leaves, small fruit, presence of stipules and strength of propagation. The distinguishing characteristics that make this cultivar unique from all other existing varieties of Prunus are, vigor, the ability to propagate well by stool beds, resistance to drought, cold and heat and the ability to serve well as rootstock that is compatible with peach and plum.

‘VSV-1’ was first asexually propagated in 1977 by the inventor at the Breeding Station in Krymsk, Russia. The method used was softwood cuttings in vitro. The distinguishing traits have been determined stable and are reproduced true to type in successive generations.

### SUMMARY OF THE INVENTION

The following traits have been repeatedly observed and represent the distinguishing characteristics of the new cultivar. These traits in combination distinguish ‘VSV-1’ from all other existing cultivars of Prunus. VSV-1 has not been tested under all possible conditions and phenotypic differences may be observed with variations in environmental, climatic, and cultural conditions, however, without any variance in genotype.

1. Prunus ‘VSV-1’ propagates well by stool beds in addition to other methods.

2. Prunus ‘VSV-1’ serves well as rootstock that is compatible with peach and plum.

3. Prunus ‘VSV-1’ is resistant to drought, cold and heat.

4. Prunus ‘VSV-1’ is strong and vigorous.

5. Prunus ‘VSV-1’ is hardy to minus 15° Centigrade.

### BOTANICAL DESCRIPTION OF THE PLANT

The following is a detailed botanical description of the new rootstock variety Prunus ‘VSV-1’. Observations, measurements, values, and comparisons were collected in McMinnville, Oreg. from the inventor. The foliage, flower and fruit exhibited by this cultivar are of no economic or commercial value, therefore comparisons and botanical descriptions of the foliage fruit and flower are made for identification purposes only. Mature specimens, as well as bareroot specimens were unavailable for photographing, at the time this document was written. The color determinations are in accordance with the RHS Colour Chart of The Royal Horticultural Society, London England except where general color terms of ordinary dictionary significance are used.

Botanical classification: Prunus ‘VSV-1’.

Parentage: Prunus ‘VSV-1’ is an induced hybrid that resulted from crossing the following plants.

Female parent.—Prunus incana (not patented).

Male parent.—Prunus tomentosa (not patented).

Type: Deciduous tree.

Use: ‘VSV-1’ serves as rootstock that is compatible with peach and plum.

Soil: Light, sandy soil required.

Light: Full sunlight.

Fruit bearing: Moderately fruit bearing (fruit in third leaf after planting).

Crop time: Requires 3 years from rooted cutting to achieve finished product size ready to ship bareroot.

Dimensions at crop time: 2.5 m in height and 2 m. in width.

Vigor: 50-60% of standard. Prunus persica is used as the standard (peach seedling).

Habit: Dwarf and generally upright with some arching branches.

Hardiness: USDA Zone 4A.

Propagation: Can be propagated by hardwood, softwood cuttings, meristem cuttings in vitro, and stool beds (layering).

Rooting habit.—Fine and fibrous initially. After 1 year roots become fleshy and thick.

Time to initiate roots.—5-6 months to develop roots at 22-25° Centigrade.

Disease and insect resistance.—Normal resistance to disease and insects.

Trunk:

Trunk dimensions.—At 3 years the trunk is 4 cm in diameter and 20 cm in height (from soil level to first branch is 20 cm).

Trunk bark surface.—Glabrous surface.

Trunk bark color.—201A.

Lenticels.—Present in small amount.

Lenticel shape.—Linear to lens shaped.

Lenticel color.—198B.

Lenticel dimensions.—Up to 4 mm. in length and 1 mm. in width.

Branches:

Branch surface.—Pubescence on young emerging branches. Absent on other branches.

Branch color.—201B.

Internode length.—5-7 cm. between nodes.

Branching angle at emergence.—60-65° and straight.

Branching habit.—Medium branching.

Pubescence.—Present on young branches.

Pubescence color.—201C.

Lenticels (quantity).—Present in small amounts on older branches and larger amount on young branches.

Lenticel shape.—Linear to lens shape.

Lenticel color.—198B.

Lenticel dimensions.—2-3 mm. in length and 1 mm. in width.

Leaves:

Arrangement.—Alternate and whorled.

Leaf length.—4.5 cm. in length.

Leaf width.—2.5-3 cm. in width.

Leaf shape.—Oblanceolate.

Leaf apex.—Acuminate.

Leaf base.—Cuneate.

Leaf color (adaxial surface).—132B.

Leaf color (abaxial surface.—132D.

Leaf surface (adaxial).—Pubescent.

Leaf surface (abaxial).—Pubescent.

Leaf margins.—Crenulate.

Leaf division.—Simple.

Petiole dimensions.—4-5 mm. in length and 1 mm. in diameter.

Petiole color.—131C.

Petiole surface.—Pubescent.

Stipules.—Present.

Stipule dimensions.—2-2.5 mm. in length and 1 mm. in width.

Stipule color.—132D.

Pubescence.—Present on adaxial and abaxial surfaces.

Pubescence color.—201D.

Venation pattern.—Pinnate.

Vein color (adaxial and abaxial surfaces).—131A.

Leaf texture.—Wrinkled, and firm to leathery texture.

Leaf strength.—Moderate strength.

Leaf appearance.—Velvety on abaxial surface and dull on adaxial surface.

Fruit:

Maturity.—70 to 75 days to mature.

Dates of picking.—Not a fruit crop. Fruit has no commercial value.

Production.—Moderately fruit bearing.

Fruit form.—Globose.

Stem dimensions.—4-5 mm. in length and 1 mm. in width.

Stem color.—143B.

Skin color.—45B.

Skin surface.—Puberulent.

Lenticels.—Absent.

Flesh color.—61D.

Flesh texture.—Juicy.

Flavor.—Sour cherry.

Aroma.—None.

Seed number.—One drupe.

Seed shape.—Round.

Seed color.—164A.

Seed dimensions.—5-6 mm. in diameter and 5-6 mm. in length.

Storage.—None has been determined.

Use.—None has been determined.

Flower:

Arrangement.—Solitary.

Flower shape.—Rotate.

Bud dimensions.—3mm. in width and 3 mm. in length

Bud color.—56D.

Time of bloom.—Flowers bloom at 1 year and last for 7-8 days.

Flower diameter.—2.5 cm. in diameter.

Flower depth (throat).—3 mm. in depth.

Bloom quantity.—50-60 flowers per branch.

Number of petals.—Five petals in number.

Fused or unfused.—Petals are unfused.

Petal shape.—Obovate.

Petal margin.—Entire and wavy.

Flower color fully opened (upper and lower surfaces).—155C.

Dimensions of peduncle.—3-4 mm. in length and up to 1 mm. in width.

Color of peduncle.—143C.

Surface of peduncle.—Pubescent.

Calyx dimensions.—3 mm. in length and 2.5 mm. in width.

Calyx surface.—Minimal pubescence.

Calyx color.—146B.

Number of sepals.—Five sepals in number.

Natural flowering season.—Spring.

Persistent or self-cleaning.—Self-cleaning.

Fragrance.—None.

Reproductive organs:

Stamen number.—Polyandrous. 12-15 stamens and unequal lengths.

Stamen color.—155C.

Anther.—Round with stamen attached at center of dorsal surface.

Anther color.—20B.

Anther dimensions.—1 mm. in length and 1 mm. in width.

Amount of pollen.—Large amount.

Color of pollen.—20B.

Pistil.—One in number.

Pistil color.—1C.

Pistil dimensions.—4 mm. in length and 1 mm. in width.

Style color.—9A.

Style dimensions.—1 mm. in length and 0.50 mm. width.

Ovary dimensions.—2 mm. in length and 2 mm. in width.

Ovary color.—155C.

Ovary position.—Superior.

1. A new and distinct rootstock variety of Prunus named ‘VSV-1 as described herein:
